(19) United States
(12) Patent Application Publication
(10) Pub. No.: US 2008/0171913 A1
(43) Pub. Date: Jul. 17, 2008

(54) Method and Apparatus for Monitoring Long Term and Short Term Effects of a Treatment

(75) Inventors: Jette Randlov, Vaerlose (DK); Jon Ulrich Hansen, Herlev (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/666,802

(22) PCT Filed: Nov. 14, 2005
(86) PCT No.: PCT/EP05/12159

§ 371 Date: Nov. 13, 2007

(30) Foreign Application Priority Data
Nov. 15, 2004 (EP) 04078132.0

(51) Int. Cl. A61B 5/00; G06F 19/00

(52) U.S. Cl. 600/300

(57) ABSTRACT

A method and apparatus for monitoring long term and short term effects of a medical treatment having a build-in dilemma between conflicting objectives are provided. A plot of the temporal development of a balance between long term and short term effects is obtained. Thereby is provided an illustrative and easy-to-use tool to express contradicting objectives and enabling a user to balance the two in a deliberate and calculated fashion. Suitable for diabetes treatment balancing the risk of long term complications against the short term risk of severe hypoglycemia.

### FIELD OF THE INVENTION

The present invention relates to a method and an apparatus for monitoring long term effects and short term effects of a medical treatment. The present invention may be used for helping a patient in adjusting a medical treatment in such a way that risks relating to long term objectives are kept as low as possible with due respect to risks relating to short term objectives and vice versa.

### BACKGROUND OF THE INVENTION

In chronic disease, there is often a balance between long term and short term side-effects of a drug, and the consequences of not taking the drug. Examples of this are given below.

In diabetes one objective is tight control to minimise the risk of long term complications, such as circulatory disturbances or diabetic retinopathy. On the other hand, avoiding hypoglycemias and the related short term hazards pose another very urgent short term objective. Hence, optimal glucose control is often not what the person having diabetes is driven to due to the associated increased risk of hypoglycemia and the immediate inconveniences related thereto for the person. It is therefore tempting for the person having diabetes to establish a safety margin in terms of an elevated target glucose level which increases the risk of long term complications, even though these long term complications may be more severe than the short term complications.

Severe asthma, treated with steroid. The (short term) risk of not taking the drug is asthma attacks. The (long term) risk of taking the drug is the side effects of the drug, i.e. iatrogenic hypercorticism, with osteoporosis, risk of fractures, cushingoid fat distribution, worsening of diabetes, psychological symptoms, etc.

Menopause, especially in case of women with increased risk of osteoporosis. In the short term, the symptoms of menopause may be severe, requiring substitution with oestrogen. This will also in the long term reduce the risk of osteoporosis. In the long term, treatment with oestrogen is a risk factor for cancer of the endometrium.

Hypertension and hypercholesterolaemia. The side effects of the drugs to be weighed against the long term risk of arteriosclerosis and cerebral damage.

It is therefore desirable to be able to balance long term objectives and short term objectives of a treatment.

WO 00/05671 discloses a method of analysing an evolution of a biological system comprising the steps of determining a series of variables upon which a state of the biological system depends, mapping the variables to an n-dimensional space, and wherein the evolution of the biological system is monitored utilising a trajectory formed from sets of the variables which define the states of the biological system at different times, thereby using time as a parameter in the n-dimensional space in a manner such that every point on the trajectory corresponds to at least one value of time.

WO 01/13786 describes a method and apparatus for predicting the risk of hypoglycemia. The method utilizes blood glucose (BG) sampling, insulin/injection records, heart rate information and heart rate variability information to estimate BG in the near future and to estimate the onset of hypoglycemia. However, the method and the apparatus disclosed in WO 01/13786 do not help the person having diabetes in balancing the treatment in order to minimise long term and short term complications.

WO 01/72208 describes a method, system, and computer program product being directed to predicting the long term risk of hyperglycemia, and the long term and short term risks of severe hypoglycemia in diabetes, based on blood glucose readings collected by a self-monitoring blood glucose device. An intelligent data interpretation component is introduced which is capable of predicting both HbA_{1c} and periods of increased risk of hypoglycemia. Based on these predictions the diabetic can take steps to prevent the adverse consequences associated with hyperglycemia and hypoglycemia.

None of the references mentioned above describe an illustrative and easy-to-understand tool for guiding a person having a disease with conflicting long term and short term objectives of the corresponding treatment in order to balance these long term and short term objectives to obtain an optimum treatment for the person. Furthermore, the prior art references do not disclose a tool for balancing the treatment over a longer period of time.

### SUMMARY OF THE INVENTION

It is, thus, an object of the present invention to provide an illustrative and easy-to-understand tool as described above.

It is a further object of the present invention to provide a method and an apparatus which helps a person to balance a treatment between long term objectives and short term objectives of the treatment in order to avoid long term complications as well as short term complications or inconveniences to the greatest extent possible.

It is an even further object of the present invention to provide a tool for balancing the treatment of a disease between long term and short term objectives over a longer period of time.

According to a first aspect of the present invention, the above and other objects are fulfilled by providing an apparatus for monitoring long term and short term effects of a medical treatment of a human or animal body, the apparatus comprising:
means for defining a treatment parameter of the body, which is susceptible to influence of the medical treatment, and for defining one or more predetermined intervals of values of the treatment parameter in such a way that values within the predetermined interval(s) are known to have larger significance with respect to short term effects of the medical treatment than values outside the predetermined interval(s),
means for providing data including a plurality of values of said treatment parameter,
means for processing said data, the processing means comprising:
   means for obtaining an authentic mean value using the data,
   means for applying a mathematical transformation to each of the values in the data to obtain transformed values,
   means for obtaining a non-authentic mean value using the transformed values, said mathematical transformation influencing the transformed values in such a way that values in the data, which are within the predetermined interval(s), have more significant influence on the non-authentic mean value than on the authentic mean value,
means for plotting said authentic and non-authentic mean values as a point in a two-dimensional representation, said point thereby representing a balance between long term effects and short term effects of the medical treatment, and
means for displaying a temporal development of said balance between long term effects and short term effects of the medical treatment.

In case the treatment is a diabetes treatment, the means for providing data may advantageously comprise a blood glucose (BG) measurement apparatus. Alternatively, the means for providing data may comprise a sphygmomanometer (in case it is desired to measure blood pressure), and/or any other suitable kind of measuring apparatus being adapted to measure the desired kind of treatment parameter values.

Alternatively, the means for providing data may comprise means for communicating with an external device being adapted to measure the desired kind of treatment parameter values, e.g. any of the devices mentioned above. In this case the actual measurements are performed using a separate apparatus which may be permanently or temporarily connected to the apparatus of the present invention. The data may be communicated to the apparatus of the present invention using a wired connection, such as a network cable, a wireless connection, such as a Local Area Network (LAN) connection, an infrared connection, a radio frequency (RF) connection, a Blue Tooth® connection, or any other suitable kind of connection. Alternatively, the external device may be a computer device which has previously obtained the data from a measuring device.

The processing means may comprise a personal computer (PC). Thus a PC may form part of the apparatus of the present invention. Alternatively, the apparatus may be connected to a PC which performs all the processing.

The apparatus may form part of a drug delivery device, such as a syringe device, e.g. a doser pen, or a pumping device. Alternatively, the apparatus may be adapted to communicate with a drug delivery device. Thus, in case it is determined that an adjustment of the treatment is necessary in order to maintain a balance between the long term and short term objectives, this information may be provided directly to the drug delivery device.

For example, a BG measurement apparatus, processing means and a display screen may be integrated into a doser pen for delivering a dose of insulin. Alternatively, one or more of these devices may be separate, but adapted to communicate with one or more of the other devices.

The displaying means may comprise at least one of a personal digital assistant (PDA), a personal computer (PC), a mobile phone and a medical device. Thus, the temporal development of the balance may be displayed on any one of these devices. The apparatus may form part of the device(s) in question. Alternatively, the apparatus may be adapted to communicate with one or more of the devices. It is advantageous that the development can be displayed on a portable device, because it makes it possible for the person having the disease to easily monitor the treatment regardless of where the person is. It is also advantageous that the development can be displayed on a PC because this opens the possibility of performing further processing of the results, e.g. statistics, because the processing capacity of a PC is normally somewhat larger than the processing capacity of a portable device. Furthermore, a monitor for a PC is normally larger than a monitor for a portable device, and it may therefore be possible to see more details of the plot on a PC.

The medical device may, e.g., be a drug delivery device or a measuring device for measuring one or more medical parameters.

The apparatus may further comprise means for printing at least the temporal development of the balance. The printing means may, e.g., form part of one of the devices mentioned above. Thus, the development in time of the plot may be printed from a PC, a PDA, etc. Alternatively, the printing means may form part of the apparatus, or the apparatus may be adapted to communicate directly with a printer.

According to a second aspect of the invention, the above and other objects are fulfilled by providing a method for monitoring long term and short term effects of a medical treatment of a human or animal body, the method comprising the steps of:
defining a treatment parameter of the body, which is susceptible to influence of the medical treatment,
defining one or more predetermined intervals of values of the treatment parameter in such a way that values within the predetermined interval(s) are known to have larger significance with respect to short term effects of the medical treatment than values outside the predetermined interval(s),
providing first data including a plurality of values of said treatment parameter, the plurality of values of said treatment parameter having been obtained at first points in time,
using the values of the first data to obtain a first authentic mean value,
applying a mathematical transformation to each of the values in the first data to obtain first transformed values,
using the transformed values to obtain a first non-authentic mean value, said mathematical transformation influencing the transformed values in such a way that values in the first data, which are within the predetermined interval(s), have more significant influence on the non-authentic mean value than on the authentic mean value, whereby it is achieved that:
   short term effects of the medical treatment are more strongly reflected by the non-authentic mean value than by the authentic mean value, and
   long term effects of the medical treatment are more strongly reflected by the authentic mean value than by the non-authentic mean value,
plotting said first authentic and non-authentic mean values as a point in a two-dimensional representation, said point thereby representing a balance between long term effects and short term effects of the medical treatment as provided by the first data,
   the method further comprising the steps of:
providing second data including a plurality of further values of said treatment parameter, the plurality of further values having been obtained at second points in time, and using the values of the second data to obtain a second authentic mean value,
applying said mathematical transformation to each of the values in the second data to obtain second transformed values, and using the second transformed values to obtain a second non-authentic mean value,
plotting said second authentic and non-authentic mean values as a further point in said two-dimensional representation,
   whereby said points in the two-dimensional representation provide a plot of temporal development of the balance of long term and short term effects of the medical treatment.

It should be noted that a skilled person would readily recognise that any feature described in connection with the first aspect of the invention can also be combined with the second aspect of the invention, and vice versa.

In case the medical treatment is a diabetes treatment, the treatment parameter may advantageously be a physiological parameter, such as blood glucose (BG). Alternatively, the treatment parameter may be a medical parameter, such as insulin consumption over a period of time. In case of any other disease with build-in dilemmas, e.g. one of the diseases mentioned above, a suitable treatment parameter which is susceptible to influence the medical treatment for that disease may be used.

The predetermined interval(s) of values of the treatment parameter values is/are defined in such a way that values within the predetermined interval(s) are known to have larger significance with respect to short term effects of the medical treatment than values outside the predetermined interval(s). The predetermined interval(s) may be just one interval, e.g. positioned at one end of a range in which it can normally be expected to measure the treatment parameter, e.g. very high values or very low values. Alternatively, it may be an interval positioned somewhere in such a range, e.g. near the middle of the range. Alternatively, two or more intervals may be defined, distributed somehow along such a range, e.g. two intervals positioned at or near the extreme ends of such a range. The predetermined interval(s) need not be fixed interval(s). They may instead have sliding boundaries in the sense that the significance with respect to short term effects of the medical treatment may decrease as the values move away from a specific point. This should be appropriately reflected by the mathematical transformation, i.e. the most significant values should be more strongly enhanced than values having less significance. Furthermore, the predetermined interval(s) may vary from one person to another.

The steps of providing first and second data may, e.g., be performed by measuring the relevant treatment parameter values at certain time intervals. Such measurements may advantageously be performed by the person having the disease, i.e. in a self-monitoring way. Alternatively or additionally, the data may be provided from a data storage device which has obtained the data from a measuring device.

The provided data is processed in order to obtain processed values being indicative of the present balance between long term effects and short term effects of the medical treatment. This is done in two steps.

An authentic mean value is obtained using the values of the first/second data. By ‘authentic mean value’ is, thus, meant a mean value obtained directly on the basis of the values of the provided data.

Furthermore, a mathematical transformation is applied to each of the values in the first/second data, thereby obtaining first/second transformed values. Using these transformed values, a non-authentic mean value is obtained. By ‘non-authentic mean value’ is meant a mean value which is obtained on the basis of transformed values, i.e. the values have been ‘manipulated’ before the mean value is obtained, as opposed to the authentic mean value which was obtained directly from the values. The mathematical transformation influences the values in such a way that values within the predetermined interval(s), i.e. values being known to have relatively large significance with respect to short term effects, are transformed into transformed values which have a more significant influence on the non-authentic mean value than the remaining transformed values.

It is therefore achieved that short term effects of the medical treatment are more strongly reflected by the non-authentic mean value than by the authentic mean value, and long term effects of the medical treatment are more strongly reflected by the authentic mean value than by the non-authentic mean value.

The authentic mean value and the non-authentic mean value may be regarded as two coordinates, and they may therefore be plotted as a point in a two-dimensional representation. Such a plotted point represents a balance between long term effects and short term effects of the medical treatment.

In case the disease is diabetes and the treatment parameter is blood glucose (BG), the authentic mean value of the BG level will give an indication of the risk of long term complications, since a high mean BG value increases the risk of long term complications. Similarly, the non-authentic mean value will indicate the risk of short term complications, such as severe hypoglycemia.

Repeating the method described above results in a plot of temporal development of the balance of long term and short term effects of the medical treatment. Looking at such a temporal plot a person, e.g. the person receiving the medical treatment, will know whether or not the treatment will need adjustment in order to provide an optimum balance between the long term and short term objectives of the treatment, or if there is room for improvement, in which case the person may decide to adjust the treatment.

Thus, the temporal plot provides a tool for the person for evaluating the trend of the plotted points. Looking at the temporal plot the person may very quickly determine whether or not the balance is relatively stable or it moves, slowly or quickly, towards undesired regions. Such information may be very important in relation to whether or not a person chooses to adjust the treatment.

The plot may be in the form of a two-dimensional coordinate system with the authentic mean value (i.e. the risk of long term complications) shown along one axis and the non-authentic mean value (i.e. the risk of short term complications) shown along the other axis. In this case the person would normally like to keep the processed value near a centre point since this would imply minimum risks for long term as well as short term complications.

Alternatively, the plot may be in the form of a ‘road’ with an optimum value illustrated in the middle of the road and the highest/lowest acceptable values shown as the edges of the road. The edges should not be exceeded, and the person should attempt to keep the value at or near the middle, thereby aiming at an optimum balance.

Furthermore, in any of the above examples, the plot may be made even more illustrative and helpful by adding colours to the plotted values, the colours being indicative of the present status, e.g. red signalling a high risk, yellow signalling a medium risk and green signalling a low risk.

Thus, an illustrative and easy-to-understand tool has been provided which expresses the contradictive objectives and helps a person in balancing long term objectives and short term objectives of a treatment in a deliberate and calculated fashion.

The first authentic mean value may be obtained by calculating a weighted average of the values of the first data, and the second authentic mean value may be obtained by calculating a weighted average of the values of the second data. In particular, it may be just a simple average, i.e. all the weights are equal to 1. Alternatively, the weights may vary according to the value, the time of day the value is obtained, how long time has elapsed since the value was obtained, and/or according to any other suitable criteria.

Thus, the weighted averages may be calculated using the formula:

$${TP}_{mean} = \frac{2}{N ( N - 1 )} {\sum }_{i = K + 1}^{K + N} {TP}_{i} \cdot ( i - K ) ,$$

wherein TP_{K} is the most recent value of the treatment parameter, and N is the number of values in the first/second data. When using this formula, most weight is given to the most recent treatment parameter values of the first/second data, thereby giving most weight to, e.g., most recent measurements.

Similarly, the first non-authentic mean value may be obtained by calculating a weighted average of the first transformed values, and the second non-authentic mean value may be obtained by calculating a weighted average of the second transformed values.

Thus, the weighted averages may, in this case, be calculated using the formula:

$$Tranformed {TP}_{mean} = \frac{2}{N ( N - 1 )} {\sum }_{i = K + 1}^{K + N} Transformed TP ( {TP}_{i} ) \cdot ( i - K ) ,$$

wherein TP_{K} is the most recent value of the treatment parameter, and N is the number of values in the first/second data. Again, when using this formula, most weight is given to the most recent transformed treatment parameter values of the first/second data.

The steps of applying a mathematical transformation may be performed in such a way that each transformed value is larger than 0. This is an advantage, because thereby all treatment parameter values of the data are taken into consideration. This provides a better basis for issuing a ‘warning’ in case it is necessary to adjust the treatment.

Alternatively or additionally, the steps of applying a mathematical transformation may be performed in such a way that lowering the value of the treatment parameter by 1 unit results in the corresponding transformed value being doubled. Thus, the transformation may be an exponentially decreasing function. This is an advantage because it provides the possibility of, in an easy manner, giving low values of the dataset high priority or weight when the non-authentic mean value is subsequently obtained. In case the disease is diabetes and the treatment parameter values are BG values, this is particularly advantageous, because very low BG values should be taken very seriously in order to prevent hypoglycemia.

The mathematical transformation applied may be of the form:

$$Transformed value = \frac{{a}^{( b - TP )}}{c} ,$$

wherein a, b and c are real constants, and TP is the value of the treatment parameter, e.g. a transformation of the form:

$$Transformed value = \frac{{2}^{( 8 - TP )}}{1.28} .$$

As mentioned above, the treatment may be a diabetes treatment, in which case the treatment parameter may advantageously be blood glucose (BG). Alternatively, the treatment may be treatment of severe asthma with steroids, treatment of menopause with oestrogen or treatment of hypertension and hypercholesterolaemia. Alternatively, the treatment may be any other suitable kind of treatment having a build-in dilemma of long term objectives and short term objectives, thereby requiring a balancing of these objectives.

### BRIEF DESCRIPTION OF THE DRAWINGS

The invention will now be described with reference to the accompanying drawings in which

FIG. 1 shows one kind of plot obtained using the present invention, and

FIG. 2 shows another kind of plot obtained using the present invention.

### DETAILED DESCRIPTION OF THE DRAWINGS

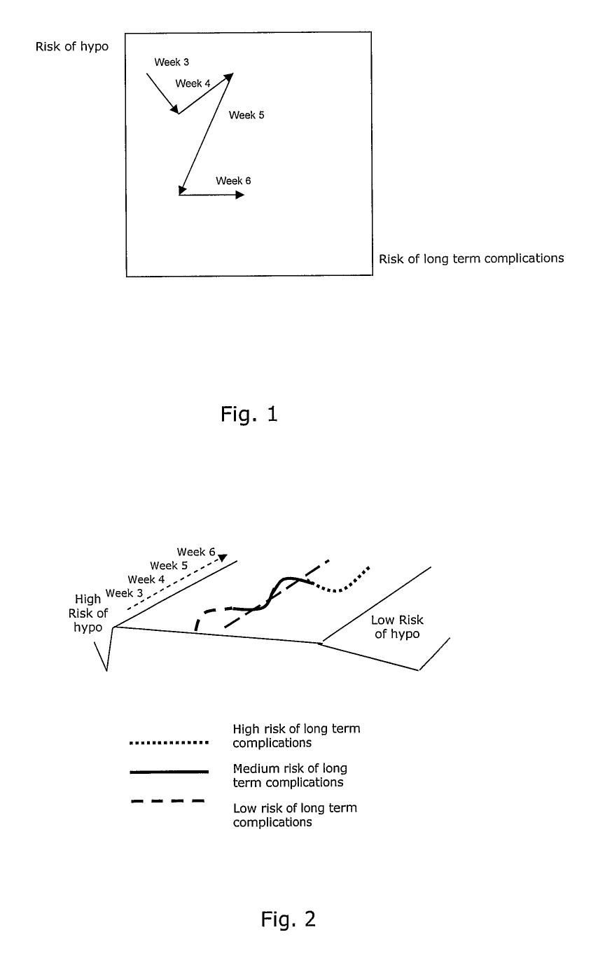

FIG. 1 shows a two-dimensional plot related to diabetes treatment of a person. Along the first axis the risk of long term complications related to a high BG value is shown, the risk increasing when moving to the right in the plot. The value of the first axis is an authentic mean value of measured BG values. Along the second axis the risk of short term complications, i.e. hypoglycemia, is shown, the risk increasing when moving upward in the plot. The value of the second axis is the non-authentic mean value of transformed BG values.

Thus, in the plot shown in FIG. 1, the non-authentic mean value is plotted against an authentic mean BG value. In the ideal situation the values of the plot should be in the lower left corner of the plot, indicating a low risk of short term complications as well as a low risk of long term complications. Similarly, the values should not be in the upper right corner of the plot. If values are changing over time, it is most desirable that these chances result in movements in the plot along with or parallel to the diagonal connecting the upper left corner and the lower right corner. This ensures that the person remains within a range where long term objectives and short term objectives are traded off against each other, and that the ‘well-being’ of the person is not changed considerably during the change in values. On the other hand, if changes result in movements in the plot which are substantially perpendicular to the diagonal mentioned above, the person will sometimes be doing well and sometimes be doing badly. This is not good for the general well-being of the person and should therefore be avoided. Therefore, if this kind of development is detected, the person should react by considering adjusting the treatment.

FIG. 1 also shows plots from a person relating to four weeks of measurements. The plots corresponding to the weeks are labelled ‘week 3’, ‘week 4’, ‘week 5’ and ‘week 6’, respectively. The plot thereby shows the development during these four weeks of the authentic and non-authentic mean values for this person. As can be seen, the person started out with a high risk of short term complications in return for a very low risk of long term complications. During week 3 the risk of short term complications has become lower at the expense of a slightly increased risk of long term complications. During week 4 the risk of long term complications as well as the risk of short term complications have increased. This is very bad and should make the person consider whether an adjustment of the treatment is needed. During week 5 the risk of long term complications as well as the risk of short term complications have been lowered considerably, possibly due to an adjustment of the treatment. During week 6 the risk of long term complications is increased without the risk of short term complications decreasing. This might also call for an adjustment of the treatment, but since the risk of long term complications is not alarmingly high, the person may also choose to maintain the current treatment for the time being.

FIG. 2 shows another plot in the form of a ‘road’. The middle of the road (dashed line) indicates an optimum value of the non-authentic mean value. Time increases along the road as indicated by the dashed arrow to the left of the road. The authentic and non-authentic mean values vary across the road. The plane part of the road indicates a range in which the values should be allowed to be. The slope on the right side of the road indicates an area of low risk of hypoglycemia, i.e. short term complications, and the (steeper) slope on the left side of the road indicates an area of high risk of hypoglycemia. The plot of various line styles on the road represents the development in time of the authentic mean value. Each line style represents a ‘risk regime’ of long term complications. Thus, the dotted line represents a high risk of long term complications, the solid line represents a medium risk of long term complications, and the dashed line represents a low risk of long term complications. During the time period represented in the plot, the person has moved from low risk of long term complications over medium risk to high risk of long term complications. At the same time, the person has maintained a risk level of short term complications which is within an acceptable range.

The plots shown in FIGS. 1 and 2 both provide a valuable tool for a person having a disease with in-build dilemmas between conflicting objectives for balancing these conflicting objectives. The person can readily see if an adjustment of the treatment may be necessary. Furthermore, the plots of FIGS. 1 and 2 both provide the person with information relating to the development in time of the plotted values, and this is an important tool when balancing the treatment between long term and short term objectives.

1. An apparatus for monitoring long term and short term effects of a medical treatment of a human or animal body, the apparatus comprising:
means for defining a treatment parameter of the body, which is susceptible to influence of the medical treatment, and for defining one or more predetermined intervals of values of the treatment parameter in such a way that values within the predetermined interval(s) are known to have larger significance with respect to short term effects of the medical treatment than values outside the predetermined interval(s),
means for providing data including a plurality of values of said treatment parameter,
means for processing said data, the processing means comprising:
means for obtaining an authentic mean value using the data,
means for applying a mathematical transformation to each of the values in the data to obtain transformed values,
means for obtaining a non-authentic mean value using the transformed values, said mathematical transformation influencing the transformed values in such a way that values in the data, which are within the predetermined interval(s), have more significant influence on the non-authentic mean value than on the authentic mean value,
means for plotting said authentic and non-authentic mean values as a point in a two-dimensional representation, said point thereby representing a balance between long term effects and short term effects of the medical treatment, and
means for displaying a temporal development of said balance between long term effects and short term effects of the medical treatment.

2. An apparatus according to claim 1, wherein the means for providing data comprises a blood glucose (BG) measurement apparatus.

3. An apparatus according to claim 1, wherein the processing means comprises a personal computer (PC).

4. An apparatus according to claim 1, wherein the apparatus forms part of a drug delivery device.

5. An apparatus according to claim 1, wherein the displaying means comprises at least one of a personal digital assistant (PDA), a personal computer (PC), a mobile phone and a medical device.

6. An apparatus according to claim 1, further comprising means for printing at least the temporal development of the balance between long term effects and short term effects of the medical treatment.

7. A method for monitoring long term and short term effects of a medical treatment of a human or animal body, the method comprising the steps of:
defining a treatment parameter of the body, which is susceptible to influence of the medical treatment,
defining one or more predetermined intervals of values of the treatment parameter in such a way that values within the predetermined interval(s) are known to have larger significance with respect to short term effects of the medical treatment than values outside the predetermined interval(s),
providing first data including a plurality of values of said treatment parameter, the plurality of values of said treatment parameter having been obtained at first points in time,
using the values of the first data to obtain a first authentic mean value,
applying a mathematical transformation to each of the values in the first data to obtain first transformed values,
using the transformed values to obtain a first non-authentic mean value, said mathematical transformation influencing the transformed values in such a way that values in the first data, which are within the predetermined interval(s), have more significant influence on the non-authentic mean value than on the authentic mean value, whereby it is achieved that:
short term effects of the medical treatment are more strongly reflected by the non-authentic mean value than by the authentic mean value, and
long term effects of the medical treatment are more strongly reflected by the authentic mean value than by the non-authentic mean value,
plotting said first authentic and non-authentic mean values as a point in a two-dimensional representation, said point thereby representing a balance between long term effects and short term effects of the medical treatment as provided by the first data,
the method further comprising the steps of:
providing second data including a plurality of further values of said treatment parameter, the plurality of further values having been obtained at second points in time, and using the values of the second data to obtain a second authentic mean value,
applying said mathematical transformation to each of the values in the second data to obtain second transformed values, and using the second transformed values to obtain a second non-authentic mean value,
plotting said second authentic and non-authentic mean values as a further point in said two-dimensional representation,
whereby said points in the two-dimensional representation provide a plot of temporal development of the balance of long term and short term effects of the medical treatment.

8. A method according to claim 7, wherein the first authentic mean value is obtained by calculating a weighted average of the values of the first data, and wherein the second authentic mean value is obtained by calculating a weighted average of the values of the second data.

9. A method according to claim 8, wherein the weighted averages are calculated using the formula:
$${TP}_{mean} = \frac{2}{N ( N - 1 )} {\sum }_{i = K + 1}^{K + N} {TP}_{i} \cdot ( i - K ) ,$$
wherein TP_{K} is the most recent value of the treatment parameter, and N is the number of values in the first/second data.

10. A method according to claim 7, wherein the first non-authentic mean value is obtained by calculating a weighted average of the first transformed values, and wherein the second non-authentic mean value is obtained by calculating a weighted average of the second transformed values.

11. A method according to claim 10, wherein the weighted averages are calculated using the formula:
$$Tranformed {TP}_{mean} = \frac{2}{N ( N - 1 )} {\sum }_{i = K + 1}^{K + N} Transformed TP ( {TP}_{i} ) \cdot ( i - K ) ,$$
wherein TP_{K} is the most recent value of the treatment parameter, and N is the number of values in the first/second data.

12. A method according to claim 7, wherein the steps of applying a mathematical transformation are performed in such a way that each transformed value is larger than 0.

13. A method according to claim 7, wherein the steps of applying a mathematical transformation are performed in such a way that lowering the value of the treatment parameter by 1 unit results in the corresponding transformed value being doubled.

14. A method according to claim 7, wherein the mathematical transformation applied is of the form:
$$Transformed value = \frac{{a}^{( b - TP )}}{c} ,$$
wherein a, b and c are real constants, and TP is the value of the treatment parameter.

15. A method according to claim 14, wherein the mathematical transformation applied is of the form:
$$Transformed value = \frac{{2}^{( 8 - TP )}}{1.28} .$$

16. A method according to claim 7, wherein the medical treatment is a diabetes treatment, and the treatment parameter is blood glucose (BG).
